(19) United States
(12) Patent Application Publication
(10) Pub. No.: US 2024/0391132 A1
(43) Pub. Date: Nov. 28, 2024

(54) METHOD FOR STABILISATION, HYDROPHOBATION AND ENHANCED DURABILITY TREATMENT OF RENEWABLE LIGNO-CELLULOSIC MATERIALS AND A RESULTING BIO-BASED PRODUCT

(71) Applicant: FIBERDOM OY, Helsinki (FI)

(72) Inventor: Duncan MAYES, Helsinki (FI)

(21) Appl. No.: 18/262,697

(22) PCT Filed: Jan. 25, 2022
(86) PCT No.: PCT/FI2022/050049

§ 371 Date: Jul. 24, 2023

(30) Foreign Application Priority Data
Jan. 25, 2021 (FI) 20215084

(51) Int. Cl. B27K 3/36; B27K 3/15; C08H 8/00; D21H 17/18; D21H 17/60; D21H 21/16

(52) U.S. Cl. CPC B27K 3/36; B27K 3/156; C08H 8/00; D21H 17/18; D21H 17/60; D21H 21/16; B27K 2240/70

(57) ABSTRACT

Disclosed is a method for stabilisation, hydrophobation and enhanced durability treatment of a ligno-cellulosic material. The method includes the steps of applying a cross-linking formulation including at least one cross-linking acid and at least one polyol, and hydophobation emulsion to a ligno-cellulosic material. The chemically treated material is then subjected to a temperature initiating an esterification reaction between the cross-linking acid, the hydroxyl groups of the cellulose and the polyol. Also disclosed is a bio-based material obtainable by the method.

### CROSS-REFERENCE TO RELATED APPLICATIONS

This application is the US national stage of PCT/FI2022/050049, filed Jan. 25, 2022, which claims the priority of FI 20215084, filed Jan. 25, 2021. The entire contents of each foregoing application are incorporated herein by reference.

### BACKGROUND OF THE INVENTION

### Field of the Invention

The present invention relates to a method for the treatment of renewable, ligno-cellulosic materials to enhance the stability, hydrophobicity, and durability of said material. An essentially organic, non plastic containing bio-based product obtained by the method is also disclosed.

The invention has been developed to provide a technically and economically viable replacement to a broad range of conventional fossil derived, non-renewable plastic products, films and other non-biodegradable materials, coatings and additives used to improve the stability, hydrophobicity and durability of renewable, bio-based ligno-cellulosic materials. The new technology will address the increasing demand for products and materials which are derived from fully renewable raw materials and which can be fully recycled and re-processed in a circular economy, thus reducing the amount of material ending up in landfills or the oceans, as well as the amount of pollutants or hazardous biproducts entering the environment or requiring specific capturing during the decomposition or burning process at the end of the product life cycle.

### Description of the Related Art

Ligno-cellulosic materials, herein also including processed ligno-cellulosic materials, such as wood, veneer, cardboard, paper, cotton, natural fibres, regenerated cellulose, and the like are as such fully renewable and recyclable bio-based materials. An inherent challenge with these is the continued durability of the material when used in moist or varying humidity environments or in applications where the material may come into contact with high humidity, moisture, liquids or grease. When exposed to such conditions for a prolonged time, the material will hold moisture and increase the risk of microbial growth. Porous and hydrophilic materials, such as paper, cardboard, fibre and particle board, and the likes will also lose its internal strength and often no longer provide the needed functionality it was intended for. To address these limitations, barriers or additives are often applied to ligno-cellulosic materials to prevent or limit the negative impacts of moisture and grease. Traditionally these are non-renewable materials containing paints, coatings, resins and adhesives, such as linear low-density polyethylene (LLDPE), PVC, PE, Phenolic resins, Isocyanate based resins, metal-based wood preservatives and the likes. The use of a such coatings, binders and additives increases the complexity of the recycling and reprocessing of the product as the very different materials often need to be carefully separated before re-processing of the material is possible. Bio-based alternatives to coating agents and additives prolonging the lifespan and diversifying usage of ligno-cellulosic materials are therefore of high interest.

The present invention provides a method for treatment of ligno-cellulosic materials enhancing its strength and durability properties and/or moisture and liquid resistance without the use of films, coating agents, binders or additives which may limit the recyclability of the product. In addition to being fully recyclable with other ligno-cellulosic materials and products the bio-based material obtained by the method shows improved anti-microbial properties.

### PRIOR ART

The use of the combination of citric acid or similar carboxylic acids and sorbitol or similar polyols as a base formulation to facilitate an esterification reaction with the hydroxyl groups within the acid and also within wood fibres is well known and at it earliest incorporated to a patent U.S. Pat. No. 3,661,955 (A) with the title “Polyesters of citric acid and sorbitol” having a priority date of 3 Nov. 1969.

Similar cross-linking reactions have later been used on different cellulosic and wood-based materials to improve the strength and durability of these, while no reference could be found to methods including a functional emulsion in the treatment process improving the hydrophobicity and thus achieving further enhanced stability and durability of said material.

The novel invention relates to a method for stabilisation, hydrophobation and enhanced durability treatment of ligno-cellulosic materials as well as a resulting bio-based product, being functionalised essentially through the cross section or having a functionalised surface layer, the product or the layer thus being of a bio-composite material. The invention is based on a novel combination of a cross-linking reaction of ligno-cellulosic materials known from prior art and a simultaneous or subsequent hydrophobation and curing reaction.

The treatment, which includes a cross-linking reaction, may be carried out on a wide range of renewable ligno-cellulosic materials. Materials containing cellulosic fibres or cellulosic pulp of different origin with available and reactive hydroxyl groups are suitable for the process.

An esterification process providing increased stiffness of the material is carried out by use of a base chemical solution containing a reactive cross-linking acid, preferably a tricarboxylic acid, and a water-soluble polyol containing multiple hydroxyl groups. The ratio of the tricarboxylic acid to polyol and the solid's ratio to the base solvent are varied depending on the end application and the desired properties of the obtained bio-based product. A carboxylic acid having at least three carboxyl groups is preferred. The polyol preferably has at least six hydroxyl groups.

The further enhanced strength properties and moisture and liquid resistance of the end product in accordance with the present invention is achieved by addition of a hydrophobation emulsion in combination with a final curing step.

The hydrophobation emulsion comprises organic and commercially available substances with hydrophobic functionality mainly derived from essential methylene groups forming a nonpolar moiety of the molecule. The emulsion is preferably formed in a base solvent such as water or an organic solvent with similar functionality, such as alcohols. A non-ionic surfactant may be added as an emulsifying agent.

The cross-linking formulation and the hydrophobation emulsion are synthesised separately at temperatures that enables formation of a solution or an emulsion of the active agents, often a temperature of around 60° and higher is beneficial. At instances where the hydrophobation additives are in a liquid form, synthesising at low temperatures may be preferable. The obtained reaction formulations are applied to the ligno-cellulosic material to be treated either as a blend or as separate formulations using methods known in the art, such as by submersion, spraying or impregnation. The uptake of the reaction formulation can be enhanced by use of heat, and for example by use of microwave treatment. Any excess formulation is extracted and may be re-used in the process.

The cross-linking formulation comprising at least one cross-linking acid and at least one polyol as well as a novel functional emulsion is applied to the ligno-cellulosic material to be treated. The cross-linking formulation and the hydrophobation emulsion may be blended prior to application onto the ligno-cellulosic material or these may be added separately, optionally using different techniques. Additional hydrophobation emulsion may also be applied to the surface of the material treated with the mixed formulation. The chemically treated ligno-cellulosic material is then subjected to a temperature range between 50° C. and 119° C. initiating evaporation of the excess moisture before further increasing the temperature to initiate an esterification reaction between the cross-linking acid, the hydroxyl groups of the cellulose and the polyol. The temperature range of the surrounding may also be broader than the above range, as the temperature within the substrate defines the initiation of a cross linking reaction. Higher drying temperatures may be used for a quicker drying step. Since the thermal energy at this stage mainly is used to convert moisture into vapor, the temperature of the substrate would still remain below the esterification temperature for a period of time that is dependent on the moisture content and the heat transfer properties of the substrate. When the heating or drying step is short enough not to rise the temperature within the substrate above 120° C., the temperature of the surrounding may be higher than the esterification temperature.

The ligno-cellulosic material to be treated in the method of the invention may be in the form of a wood veneer or glued wood veneer material, solid wood material, non-woven particle or fibre sheet material, including processed materials, such as pulp, stranded wood or other ligno-cellulosic non-woven material web or sheet such as but not limited to hemp, flax, palm, bamboo and other grass like plants and the likes. Especially preferable are treatment of pre-shaped or final products produced from such ligno-cellulosic materials or processed ligno-cellulosic materials. The obtained bio-based material is a fully organic, non plastic containing material, provided that the substrate used is a fully bio-based material. The method of the invention is, however, also suitable for ligno-cellulosic materials containing conventional glues and the likes, whereby the bio-based material obtained may be only an essentially organic, non-plastic bio-composite.

Any solid ligno-cellulosic material or combined material of similar density, such as solid wood, glued wood veneer and medium-density or high-density fibre and particle board is preferably cut or otherwise formed into a desired shape before application of the cross-linking and hydrophobation reagents, as the treatment for such materials may be an envelope treatment or a treatment through the cross section of the ligno-cellulosic substrate. The density of such a solid ligno-cellulosic material or combined material of similar density is preferably in the range from 250-1000 kg/m³. For lower density and more porous materials or substrates, such as cellulosic pulp, pulp sheets, fibres or strands of plant-derived materials, and finely chopped wood and sawdust, as well as products produced from such materials, the cross-linking formulation or the mix of the crosslinking formulation and the hydrophobation emulsion is preferably applied throughout the material. Such porous materials may be pressed and cut into final shape and, when not included in the previous chemical treatment, subjected to the hydrophobation treatment with the hydrophobation emulsion before final curing fixating the cross-linking and hydrophobation agents within the material. Sheetlike materials may be arranged in a cross-layer formation, whereby the predominantly single direction oriented fibres of one sheet are turned in a different direction in the next layer, preferably in a 90° angle, for further increased stability.

Preferable end products are different building elements, automotive parts, surface protection products and packing, storage and transportation products. Due to the non-toxic characteristics of the reagents and raw material, the resulting bio-based material, thereby also being a bio-composite material, is well suited for, but not limited to, end-use in food contact applications. Other preferred applications are in building industry and transport industry. Since no volatile solvents or hazardous substances have been used in the process, the health-risks in connection to the material and the final products are minimal, provided that the untreated substrate does not contain harmful substances. Consequently, the method of the present invention is well suited for surface treatment of materials or manufacturing of products intended for indoor use as well as closed outdoor spaces that may be subjected to varying temperature and humidity conditions. One great benefit of the material obtained by the process of the present invention is that it has a very low environmental impact upon recycling. A further preferred application is thereby the packaging industry, as materials disposed after single use is another preferred embodiment of the invention. The material treated may be paper, cardboard, or wrapping material and other ligno-cellulosic materials and products. Especially preferred is treatment of ligno-cellulosic material that is pre-formed, shaped and cut into products produced of ligno-cellulosic materials. Such products may be pre-shaped or cut paper and cardboard material as well as pre-shaped solid or medium-density or high-density ligno-cellulosic substrates. The products treated may also be in final product shape. When products of final shapes are treated only partially, the functionalised layer of the treated substrate may completely protect any untreated material beneath from humidity and moisture. Alternatively, the solid ligno-cellulosic substrate may be treated essentially through the entire cross-section may show improved resistance to humidity and moisture in its entirety.

### SUMMARY OF THE INVENTION

The object of the invention is a method for stabilisation, hydrophobation and enhanced durability treatment of a ligno-cellulosic material or product. In the treatment process, a cross-linking formulation comprising at least one cross-linking acid and at least one polyol, and a hydophobation emulsion is applied to a ligno-cellulosic material. The chemically treated material is finally subjected to a temperature initiating an esterification reaction between the cross-linking acid, the functional hydroxyl groups of the ligno-cellulosic material, such as the hydroxyl groups of the cellulose or the other primary natural polymers of the substrate, namely hemicellulose and lignin, and the polyol. The cross-linking reaction is thus fixating the hydrophobation agent within the cross-linked structure.

The crosslinking acid used in said method are selected from a range of carboxylic acids having at least two carboxyl groups. Preferably the cross-linking acid is selected from 1-hydroxypropane-1,2,3-tricarboxylic acid, propane-1,2,3-tricarboxylic acid, 2-hydroxynonadecane-1,2,3tricarboxylic acid, 2-hydroxypropane-1,2,3-tricarboxylic acid, benzene-1,3,5-tricarboxylic acid and prop-1-ene-1,2,3-tricarboxylic acid. The at least one polyol is preferably selected from xylitol, sorbitol and erythritol.

In one preferred embodiment, the hydrophobation emulsion used in the method of the present invention comprises at least one hydrophobic agent including at least one substance selected from fatty acid esters, fatty alcohols and pentacyclic triterpenoids, such as oleanolic acid, betulin and betulinic acid. In a further preferred embodiment, the at least one hydrophobic agent is selected from a range of natural oils and waxes, preferably the hydrophobic agent is carnauba wax.

The cross-linking formulation and the hydrophobation emulsion may be blended prior to application onto the ligno-cellulosic material. Alternatively, the cross-linking formulation and the hydrophobation emulsion are added separately, optionally using different techniques. Furthermore, the cross-linking formulation, the hydrophobation emulsion or the mix thereof may be applied in liquid form, preferably by submersion, spraying or curtain coating, or in a pressurised environment through impregnation using conventional pressure impregnation methods know in the art or as a mist in semi-gaseous or atomised form within a pressurised environment. The cross-linking formulation, the hydrophobation emulsion or the mix thereof may be applied to the ligno-cellulosic material at a temperature from 20, 30 or 60° C. to 119° C., more preferably from 80° C. to 100° C., even more preferably from 90° C. to 95° C. In a further preferred embodiment, the cross-linking formulation, the hydrophobation emulsion or the mix thereof is applied to the ligno-cellulosic material under microwave treatment, or the uptake is enhanced by use of microwave treatment.

The method for treating solid or similar density products is especially preferred for treatment of products or material intended for long-term use, such as construction materials.

A further object of the invention is a product of ligno-cellulosic material, the ligno-cellulosic material comprising moieties of at least one polyol and at least one organic cross-linking acid being at least partially cross-linked to the cellulose structure of a ligno-cellulosic material and/or the other natural polymers of the ligno-cellulosic material through ester bonds and wherein a hydrophobic agent is present within the ligno-cellulosic material showing a cross-linked structure. A ligno-cellulosic product of solid ligno-cellulosic material or similar density ligno-cellulosic material combined into a composite is disclosed and claimed. Since the functionalised ligno-cellulosic material thus obtained contains only bio-based crosslinking agents, it is a fully bio-based material and a bio-composite. In a preferred embodiment, said at least one hydrophobic agent includes at least one substance selected from fatty acid esters, fatty alcohols and pentacyclic triterpenoids, such as oleanolic acid, betulin and betulinic acid. In a further preferred embodiment, said hydrophobic agent is selected from a range of natural oils and waxes, preferably the hydrophobic agent is carnauba wax. A further object of the invention is a functionalised ligno-cellulosic product obtainable by a method of the present invention. An especially preferred product of solid or similar density ligno-cellulosic material is a construction material, a construction product, an interior or exterior building product, a furniture product, a transportation product or a storage product.

### BRIEF DESCRIPTION OF THE DRAWINGS

The invention is hereinafter described in detail with reference to the following drawings, wherein:

FIG. 1 is a flowchart presenting the general steps of a preferred process of the invention.

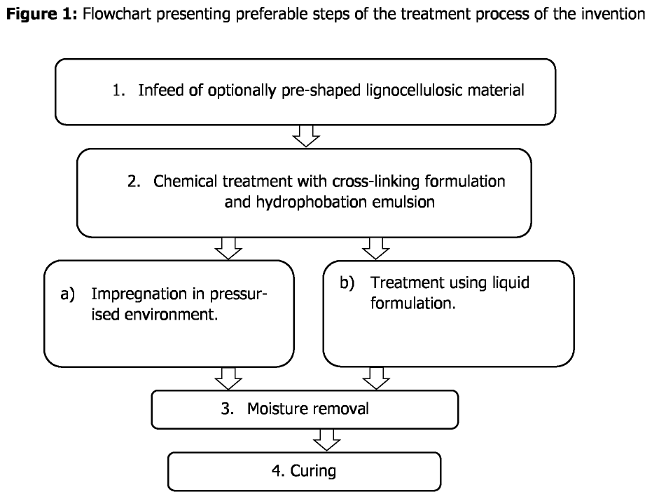

FIG. 2 is a diagram presenting the results of a soaking test carried out on pine wood samples.

### DEFINITIONS

The term ligno-cellulosic material herein refers to any kind of plant-derived material containing cellulose or cellulosic fibres, either in its natural form or in processed form. Typically, the ligno-cellulosic material contains the natural polymers lignin, hemicellulose and cellulose or cellulosic fibres. Such materials are solid wood, wood veneer, glued wood veneer, sheets or boards of non-woven particle or fibre, stranded wood or other ligno-cellulosic non-woven material, possibly in the form of webs or sheets, pulp, regenerated cellulose and the like. The ligno-cellulosic material may be derived from all living plants and the like, such as but not limited to cotton, hemp, flax, palm, bamboo and other grass like plants and the likes.

Low-weight and porous materials are considered to include materials such as cardboard, corrugated cardboard and paper. Preferably, these have been produced in industrial paper board and packaging mills utilising a broad range of raw materials as defined above. Examples of such low-weight and porous ligno-cellulosic products include folded box board products, such as folded boxes intended for short term containment of fast-food products, and corrugated box board based on, for example, brown kraft liner paper material which has been formed into a corrugated form. The products produced out of low-weight and porous material are typically short life-span products and singe-use packaging products. The products within this category are designed to be used only for a limited period and to be easily recyclable with similar raw materials. Products of low-weight and porous ligno-cellulosic material are typically used in packaging of products and goods, and are especially preferred for products and goods that are transported through varying humidity and climate regions, where high humidity may severely reduce the strength of the packaging material and where short exposure to rain may further damage the packaging. Within this application the term porous ligno-cellulosic materials refer to raw materials that before treatment according to the invention tend to disintegrate when exposed to water even after a very short period of time, such as one or two minutes. An exception is woven or non-woven fabrics, which usually have better resistance to moisture. Paper and cardboard are examples of porous materials. Further examples of products within this low-weight category are non-woven and woven fabrics. The weight of the low-weight ligno-cellulosic materials are typically 150-400 grams per m². The thickness of an individual layer of material used in such products is at the most 3 mm, or when including any void space up to 5 mm. Typically, the thickness is around 1-1.5 mm, or in the case of materials having void space, such as a in a formed corrugated box board, the thickness of the material is typically 3-4 mm.

Solid ligno-cellulosic materials and similar density ligno-cellulosic materials are within this application referring to materials consisting entirely of wood or solid ligno-cellulosic plant-derived materials as well as high-density or medium-density materials produced from solid ligno-cellulosic materials, optionally mechanically processed during the manufacturing process and combined into a composite. Examples of solid ligno-cellulosic materials and similar density materials are solid wood derived from sawmilling processes, products laminated or produced from solid wood, such as cross laminated timber (CLT), glue laminated beams and construction components and exterior and interior decorative products, wood veneers and veneer based products derived from a log peeling process, such as plywood and laminated veneer lumber (LVL) or similar, particle board, fibreboard and strand based products, which are derived from a process of combining individual ligno-cellulosic fibres or strands together through the use of adhesive and pressing technology to form a solid sheet material. All such solid ligno-cellulosic products are typically used in the construction, packaging and transport sectors. All said products may be produced from a wide range of ligno-cellulosic raw materials as defined within this application. The products considered in this category are intended for long term use, in many cases for decades, and in applications where high durability and performance is required. These products, due to the bio-based constituents, will be recyclable with no harmful emissions to the environment at the end of their useful life with other natural ligno-cellulosic construction and packaging materials. Untreated solid or similar density ligno-cellulosic material would not disintegrate when contacted with water for a prolonged time, even if swelling of the material would occur. However, some delamination may start to occur at glue lines after prolonged water exposure. The density range for solid or similar density materials is typically 250-1000 kg/m³. The thickness of a solid or similar density material is typically greater than 3 mm, more preferably greater than 4 or 5 mm.

Bio-based is herein to be understood as a material or a compound that is obtainable from a natural source or any combination of such materials or compounds. Herein the term bio-based also includes synthetically produced equivalents to such compounds and mixes consisting essentially of such compounds. The term bio-based also refers to any unprocessed or processed renewable material, especially plant-based materials.

The term recyclable herein refers to a product being recyclable together with conventional products produced from a similar material as the one treated in the process. There is no need to separate binding or functional agents prior to recycling as these are chosen from a range of bio-based agents that can be fully blended into the recycled material without significant negative effects, such as increased toxicity, formation of harmful components, formation of lumps, such as from plastic films, etc.

The term reaction formulation herein refers to the cross-linking formulation, i.e. the base formulation, the hydrophobation emulsion, i.e. the functional emulsion, or a mix of these.

The term cross-linking agent and hydrophobic agent herein refers to the active ingredient of the reaction formulation. The total solids content of cross-linking agent, i.e. the cross-linking acid and the polyol, and hydrophobic agent in the final product comprises both reacted moieties of the agents and unreacted agents in solid state.

### DETAILED DESCRIPTION

The ligno-cellulosic material used as raw material in the present invention is preferably any plant-derived material containing ligno-cellulosic or cellulosic structures that may be reacted in a cross-linking reaction. Such materials comprises a variety of wooden materials like solid wood, veneers, wood strands, wood wool, wood chips, sawdust, and wood pulp, including thermo-mechanical, chemi-thermomechanical pulp (CTMP), softwood and hardwood kraft pulps, dissolving pulp, and recycled pulp. Also processed materials or wood composites, such as laminated timber, plywood and particle boards having uncoated surfaces allowing the reaction formulation to access the cellulosic structure and the formation of ester bonds between the functional groups of the lignocellulosic material, such as the hydroxyl groups of cellulose, and the cross-linking formulation are suitable for the process. The ligno-cellulosic material may be derived from agricultural ligno-cellulosic materials such as hemp, flax, bagasse, palm, rice stems and the likes, which are also suitable for the process. It may also be carried out on cotton fibres and fibres produced from regenerated cellulose. The aim is to provide a stabilisation, hydrophobation and enhanced durability treatment encompassing at least the surface of the structure. In embodiments wherein the whole cross-section of the material is contacted with the reaction formulation, this may also function as a binder within the obtained composite material.

An especially preferred embodiment of the present invention is treatment of pre-shaped or final product shape of ligno-cellulosic material. The final curing step at a temperature initiating an esterification reaction between the cross-linking acid, the polyol and the hydroxyl groups of the ligno-cellulosic material is thus also fixating the hydrophobic agent. The substrate being a pre-shaped product includes any product of ligno-cellulosic material that is in a shape where at least one surface to be treated is formed into its final shape. Examples of pre-shaped products are wooden boards, plywood, tabletops and building materials having at least one surface of final shape, such as a smooth upper surface or other functional final shape. These products might still need cutting into desired length or similar processing prior to installation or other use. Other examples of pre-shaped or final shape substrates are cardboard products, such as transportation boxes or packing material shaped to keep an item in place. A fabric, such as a cotton fabric, treated prior to cutting and sewing is another example of a pre-shaped product. Preferable product of porous or low-weight ligno-cellulosic material according to the invention are packaging product used for a limited period of time, such as for containment of fast-food products, or for use in protecting goods when in transportation and storage where the expected lifecycle is short and material recycled, not being limited to such applications. Preferable products of solid or similar density material are construction material, furniture or transport or storage products with a long lifetime expectancy, for example five years and longer, and which can be recycled at the end of use with other ligno-cellulosic products. Examples of such products are structural wood elements like cross laminated timber, glue laminated beams, laminated veneer lumber, plywood, medium and high-density fibreboard, particle and strand boards, decking boards, flooring, cladding, wall boards, other general construction products produced from ligno-cellulosic materials, transport crates and pallets, etc.

The hydrophobation and stability treatment is thereby suitable for uncoated industrially available products having a surface of a ligno-cellulosic material containing cellulose or cellulosic fibres. The product may optionally be cut or otherwise formed into desired shape prior to the chemical treatment and the final curing step.

The ligno-cellulosic material, either in the form of raw material or in the form of a pre-shaped or final product, is fed into the treatment line. (1) For the esterification process, a base chemical solution is used. This base formulation contains at least one reactive organic cross-linking acid where one or more of the hydrogen atoms have been replaced by a carboxyl group and preferably containing at least three carboxyl groups. Preferable is use of an acid well known and approved in the food and pharmaceutical industries such as, but not limited to, 1-hydroxypropane-1,2,3-tricarboxylic acid, propane-1,2,3-tricarboxylic acid, 2-hydroxynonadecane-1,2,3-tricarboxylic acid, 2-hydroxypropane-1,2,3-tricarboxylic acid, benzene-1,3,5-tricarboxylic acid and prop-1-ene-1,2,3-tricarboxylic acid. Additionally, the chemical solution contains a water-soluble polyol which contains multiple hydroxyl groups. The polyol is preferably selected from a range of polyols obtainable from natural sources, preferably widely used and approved in the food industry such as, but not limited to, xylitol, sorbitol and erythritol. These primary components are synthesised in a base of water or similar functional organic solvent in a variety of formulated ratios between 1:1 to 5:1 cross-linking acid to polyol, in one preferred embodiment the cross-linking acid to polyol ratio is 3:1. The solids content of this base formulation is preferably between 5 and 50% by weight depending on the end application and desired properties of stiffness, bending strength and moisture resistance. The formulation is prepared at a temperature where the cross-linking acid and polyol are dissolved under stirring in the solvent used but at which a rapid esterification process is not yet initiated. A temperature ranging from 10° C. up to a temperature slightly below the pre-reaction temperature of the esterification reaction is preferred, such as a temperature range of 60-119° C. The base formulation thus obtained is herein referred to as the cross-linking formulation.

The enhanced strength properties and moisture and liquid resistance of the end product of the present invention is achieved by a curing process that may be performed using a variety of techniques. Especially for porous materials, the strength and hydrophobicity properties of the final product can be increased by performing a densifying step prior to the final curing step (4).

In order to further increase the hydrophobic properties of the final product, a functional hydrophobation emulsion is added (2). This may be blended with the cross-linking formulation and applied to the ligno-cellulosic material as a mix or may be added separately onto a material already treated with the cross-linking formulation (2, 2a, 2b) by repetition of this step or an alternative technique (2a, 2b) using only the hydrophobation emulsion.

The hydrophobation emulsion comprises organic and commercially available substances with hydrophobic functionality, often derived from essential methylene groups forming a nonpolar moiety of the molecule. Such substances functions as the hydrophobic agents, the primary constituents of which include at least one substance selected from, fatty acid esters fatty alcohols, other organic acids and hydrocarbons as well as additional or alternative functional substances selected from a range of pentacyclic triterpenoids such as, but not limited to oleanolic acid, betulin and betulinic acid. Such hydrophobic agents may be derived from natural oils and waxes, in one preferred embodiment the hydrophobic agent is carnauba wax.

The hydrophobation emulsion is preferably formed in a base solvent, possibly in combination with a non-ionic surfactant commonly used in the art for oil in water emulsions. The base solvent can be water or an organic solvent with similar functionality, such as ethanol. The cross-linking formulation and functional emulsion are synthesised separately at temperatures enabling the formation of the formulation and the emulsion. The hydrophobation emulsion may also be prepared without a base at a temperature where the hydrophobation agent is in liquid form by addition of a surfactant. The mixture is then added to the cross-linking formulation at a temperature where the hydrophobation agent is in liquid state. The cross-linking formulation may be prepared at lower temperatures, such as from 10° C. up to a pre-reaction temperature of the esterification process, often below 120° C. The aim is to apply the solution to the ligno-cellulosic substrate in a form where the esterification process of the cross-linking solution is not yet initiated, thus enabling formation of cross-linking between the cellulosic structure, or other reactive groups in the ligno-cellulosic material, and the cross-linking agents, whereby the amount of the available, moisture attracting, hydroxyl groups is reduced within the substrate. The synthetisation of the hydrophobation emulsion usually requires a temperature where the hydrophobic agent is in liquid form, for most waxes the temperature should be above 60° C. Preferable general temperatures for the preparation of the reaction formulations ranges from about 60° C. to 119° C., even more preferably from 80° C. to 100° C. The duration of this preparation step is often around 1 hour or more. As noted above, also at this stage special care should be taken not to rise the temperature to a temperature initiating a rapid esterification reaction as the aim is to introduce the cross-linking agents and the hydrophobic agent into the ligno-cellulosic material at a pre-esterification temperature, whereby an esterification reaction between the cross-linking acid and available hydroxyl groups of the cellulosic structure as well as the polyol will take place within the substrate. A pre-heated solution at a temperature of the abovementioned range has been found to increase the uptake of the reaction formulation.

The cross-linking formulation and the functional emulsion are preferably blended upon completion of the independent synthesis steps within the same or a similar temperature range. Alternatively, the functional emulsion may be applied separately to the uncured material treated with the cross-linking formulation. The ratio of the solids content of the functional emulsion to the solids content of the cross-linking formulation is preferably from about 0.1% to 15% in the mixed formulation. Preferably, the surfactant ratio of the functional emulsion ranges between 0.1% and 50% by weight of the solids content of the emulsion.

Upon final synthesising of the reaction formulations, i.e., the cross-linking formulation, the functional emulsion or the mixed formulation, the ligno-cellulosic material to be treated is exposed to the combined or separate reaction formulation via a range of alternative methods known in the art, including submersion, spraying, curtain coating or impregnation in a pressurised environment or any combination of these. The cross-linking formulation may be added to the substrate at a temperature ranging from 10° C. up to the pre-reaction temperature of the esterification reaction, preferably from about 20, 30 or 60° C. to 119° C. The hydrophobation emulsion or the premixed hydropobation emulsion and cross-linking formulation is preferably applied to the substrate at a temperature ranging from 20, 30 or 60° C. up to the pre-reaction temperature of the esterification reaction, more preferably from 80° C. to 119° C., and even more preferably from 80° C. to 100° C. Most preferred is temperatures from about 90° C. to 95° C. and from about 95° C. to 100° C. Herein, the term reaction formulation refers to the cross-linking formulation, the functional emulsion or the mix of these two prepared as described above.

The ligno-cellulosic material may be impregnated (2a) in a pressurised environment by formation of a mist of the cross-linking formulation, the functional emulsion or a mix thereof or using liquid solution. Alternatively, the cross-linking formulation, the hydrophobation emulsion or the mix thereof is applied in liquid state by any technique known in the art, such as by submersion or spray coating

The cross-linking formulation and the hydrophobation emulsion, or the mix thereof, may be applied either as a surface treatment or throughout the material to be treated. For solid and dense material, preferably in the shape of the final product or as pre-shaped elements, the process is performed as an envelope coating process. Such solid or similar density materials may have a density in the range of 250-1000 kg/m³. By at least pre-shaping the material prior to treatment, an even surface is obtained on the final product. When no cutting or other shaping is required after the treatment process, the resulting functionalised bio-based material formed around the product protects also the untreated ligno-celluosic material beneath. The thickness of the functionalised layer naturally depends on the characteristics of the ligno-cellulosic material itself, the concentration of the cross-linking formulation and the hydrophobation emulsion as well as the technique and parameters used during the application of the reaction formulation.

The water-based reaction formulations, or formulations prepared in solvents having similar properties, will make treatment of processed and porous materials very effective as the cross-linking agents and the hydrophobation agent will be carried into contact with the hydrophilic structure of the material. For porous ligno-cellulosic materials, such as a cellulosic pulp mass or sheet, non-woven wood strand or wool sheets, or when the material has been cut, chipped or stranded into smaller pieces, treatment of the whole cross-section or the material volume is often beneficial. This enables molecular interaction, and upon curing, the final esterification process to take place throughout the material, thus giving improved dimensional stability and hydrophobicity and also ensuring good interfacial properties if multiple layers of material are combined. In applications where higher flexibility of the material is needed, the reaction formulation may, however, also be applied only on the surface of the non-woven or woven ligno-cellulosic sheet, or relatively thin solid material. Likewise, this could also be achieved using a cross-linking formulation having a lower total cross-linking agent concentration or higher ratio of hydrophobic agent to cross-linking agent.

When the hydrophobation emulsion is applied separately, this may be added only onto the surface of a pre-shaped material already treated with the cross-linking formulation, such as by spray coating or curtain coating. The hydrophobation emulsion may be added during the initial treatment step and/or prior to curing of the material. An increase in contact angle of the surface of the treated material can be achieved through this additional step, making the plant based cellulosic material suitable for applications where the end product is exposed to moisture for a prolonged time or when water repellent properties are needed. Such applications are for example construction elements, furniture elements, and tabletops. Furthermore, different non-woven and woven fabrics may be treated, such as cotton fabrics or fabrics produced from regenerated cellulose. Suitable applications for these would be, for example, the use in tablecloths. The ratio of the cross-linking agent to the hydrophobation agent is chosen such that desired properties of the material is achieved.

In one embodiment where the ligno-cellulosic material is impregnated (2a) with at least one of the reaction formulations, the process may be carried out at an internal pressure between 2-10 bar and a spray release of the liquid reaction formulation to a pressure chamber to create a mist-based impregnation with gradual release of pressure to atmospheric pressure. The temperature is preferably in the range from 60° C. up to a pre-esterification temperature, where a rapid esterification reaction not yet is initiated. Usually, this temperature is around a maximum of 119° C. More preferably the temperature is between 80° C. and 100° C., even more preferably between 90° C. and 95° C. or between 95° C. and 100° C. In a further embodiment utilising the impregnation approach, the cross-linking formulation and optionally combined hydrophobation emulsion may be formed into a very fine mist by use of an atomisation technique, whereby the ready combined solution is introduced into the vacuum chamber at high velocity through suitably fine nozzles which cause the fine atomisation to a mist as it enters the chamber. This impregnation technique enables the formulation to penetrate deep into the material and is therefore especially preferred for solid wood materials, veneers, similar solid sheets, bales or rolls of cellulosic pulp sheet materials.

In another embodiment, the at least one reaction formulation is added in liquid form. (2b) Individual sheets, a volume of porous material or a piece of solid or high-density or medium-density material may be submersed in or otherwise brought in contact with the cross-linking formulation, preferably in combination with the hydrophobation emulsion. The liquid treatment may also be any kind of spray treatment known in the art, such as treatment by spray coating or curtain coating. The latter methods are also well suited for complementary hydrophobation treatment of ligno-cellulosic material already treated with the cross-linking formulation not yet cured. The temperature of the liquid formulation is preferably from 60° C. and up to the pre-reaction temperature of the esterification reaction, even more preferably from about 80° C. to 100° C. The residence time is chosen according to the intended use and the material to be treated and depends among other on the thickness and structure of the material. A residence time of 10-30 s is often sufficient for woven and non-woven ligno-cellulosic sheets, preferably being conveyed through a bath in a continuous process, or for other ligno-cellulosic materials submersed in a bath. The uptake of the solution may be enhanced by a longer residence time, such as 1 minute or more. This is preferred especially for solid materials or larger pieces of bio-based material treated by submersion. Excess liquid is then removed, for example by use of vacuum, pressing, and other techniques known in the art.

Use of microwave treatment at the point of combining the solution with the ligno-cellulosic material has been found to significantly enhance the solution uptake both in the initial treatment stage and also in the retention of the solids post drying. Use of microwave treatment to may also include microwave heating of the substrate prior to addition of the reaction formulations. Upon reaching the desired weight percentage gain (WPG) or treatment level, the ligno-cellulosic material is removed from the treatment step and, if necessary, excess solution is extracted, for example by applying pressure or via vacuum. This excess reaction formulation may be recycled for re-use. The targeted solids content to be retained in the treated portion of the ligno-cellulosic material will be determined based on the final application and controlled with residence time, temperature and possibly through regulated pressure and varying solids ratio within the solution. The targeted WPG will vary significantly depending on the substrate and the intended use of the chemically treated product and will naturally be dependent on the surface to volume ratio of the substrate. As a general approximation, the WPG in wet form may be up to around 300% for porous materials, for example in the range of 100-200%, while a WPG from, for example, 5-50% may be targeted for solid wood and similar substrates and may vary further depending on the dimensions of the object. A WPG of 200-300% in wet form corresponds approximately to a solids content of around 50-75% in the final product, but also depends greatly on the concentration of the cross-linking and hydrophobation formulation. The whole treatment process is tailored to the specific end product and intended use.

The chemically treated ligno-cellulosic material should be pre-dried after application of the separate or mixed reaction formulations to remove moisture (3) from the solvent and any absorbed humidity. This may, when necessary, be carried out for example by use of vacuum or by pressing excess liquid out of the material, such as by use of a mangle. Any excess liquid removed at this stage may be re-used in the process. The moisture content is then further reduced by evaporation. In one preferred embodiment the temperature is raised gradually, thus removing moisture slowly and simultaneously pre-heating the treated ligno-cellulosic material prior to the curing process. At the initial stage of the moisture removal process, the temperature range is preferably 50-104° C. Preferably, the moisture content is reduced to below 10% during this step, more preferably below 5%. A higher moisture content will be likely to cause deformation of the treated surface during the curing step or otherwise have a negative effect on the quality of the product. Higher moisture content may be acceptable in products not having a specific shape, such as wood wool and the like.

In practice, the step of moisture removal (3) may for example be carried out by transferring the chemically treated ligno-cellulosic material to a pre-heated oven, preferably at a temperature ranging from 50-104° C., even more preferred is a temperature of 80-100° C., where the ligno-cellulosic material is dried to a moisture content which is close to ambient with the indoor climate of the production plant. The equilibrium moisture content (EMC) in a relative humidity (RH) of 55-60% is estimated to be 7-9%. The targeted dry solids content remaining in the treated ligno-cellulosic material after drying is determined based on the end application, preferably being at least 5% by weight, often a WPG of 10-90% is targeted for applications where cross-linking formulation and additional hydrophobation emulsion is applied throughout the ligno-cellulosic material. For solid and higher density materials, such as solid wood, where only the surface layer is treated, the solids content of the reaction agents remaining in the material is naturally dependent on the surface to volume ratio of the substrate and thereby the above-mentioned percentage functions only as a reference for the solids content within said reacted layer. Porous materials, like non-woven and woven ligno-cellulosic sheets, pulp, plant-derived strands and fibres, may be formed or pressed into a desired shape after the treatment process. When layers of cellulosic materials are treated, such as non-woven sheets, these may be combined and consolidated before further forming and finally curing. Such sheets may optionally be arranged in a cross-layer formation, whereby predominantly single direction oriented fibres of one sheet are turned in a different direction in the next layer, preferably in a 90° angle.

When pressure is applied to densify or smooth out the surface of the material treated with the cross-liking formulation and the functional emulsion, a pressing temperature below the melting point of the hydrophobic agent is preferred. This reduces the mobility of the hydrophobic agent prior to the curing process finally forming a cross-linking structure that ideally fixates the hydrophobic agent within the material. During the forming and shaping process, the pressure used may range from, for example, 300 kN to 1500 kN. This further enhances the strength and hydrophobicity properties of the product, especially when formed out of chemically treated porous ligno-cellulosic materials.

Further preheating of the chemically treated ligno-cellulosic material, for example to a temperature of 90-120° C., may be carried out prior to the final curing step. This would further decrease the moisture content of the material and evacuate any absorbed humidity as well as to raise the temperature to a pre-reaction level. The pre-heating process may be applied by means of infra-red radiation, high-frequency, microwave or conventional hot air heating technologies.

The bio-based material containing cellulose is finally cured through an esterification process taking place between the at least two carboxyl groups of the cross-linking acid and the hydroxyl groups of the cellulose as well as the polyol. This reduces the amount of available hydroxyl groups of the cellulose within the substrate and forms a cross-linked structure providing improved dimensional stability within the material and enhanced durability and hydrophobicity properties.

After curing, the material shows no wax-like surface, indicating that the hydrophobic agent is at least partially stabilised or fixed within the cross-linked structure of the material and the cross-linking agents.

The curing process may be carried out by use of methods and parameters known in the art for. For solid objects and other higher density objects treated with the reaction formulation in its final shape, the curing process is preferably performed at a temperature of about 150-180° C. Depending on the thickness, the duration of the curing step may vary from about 30 min to about 2 hours.

For porous materials, a shorter reaction time may be applied. The curing temperature may also be higher, such as between 150° C. and 200° C., more preferably between 170° C. and 190° C. A curing time of between 10 to 30 minutes is often sufficient. For smaller pieces of single layer or double layer cellulosic pulp material, the curing time may be even shorter, for example 1 minute. A flexible or otherwise formable ligno-cellulosic material treated in accordance with the method of the invention may be formed prior to this final curing step by use of any known technology suitable for the material, such as stamping, rotary embossing and cutting or laser cutting.

The curing temperature of the process of the invention is preferably 120-200° C., more preferably 150-200° C., and even more preferably 150-180° C. The curing time may vary greatly depending on the temperature, and is preferably from 1, 3, 5 or 30 minutes up to 60, 90 or 120 minutes.

The method of the present invention thus results in a ligno-cellulosic composite material comprising moieties of at least one polyol and at least one organic cross-linking acid being at least partially cross-linked to the cellulose structure of the ligno-cellulosic material through ester bonds. Additionally, a hydrophobic agent is present within the bio-composite material showing a cross-linked structure. This hydrophobic agent may include at least one substance selected from fatty acid esters, fatty alcohols and pentacyclic triterpenoids, such as oleanolic acid, betulin and betulinic acid. Preferably the hydrophobation agent is selected from a range of natural oils and waxes, such as carnauba wax.

When the method of the present invention is carried out on a solid ligno-cellulosic material or similar density ligno-cellulosic material combined into a composite, the product shows at least one surface of ligno-cellulosic material comprising moieties of at least one polyol and at least one organic cross-linking acid being at least partially cross-linked to the hydroxyl groups of a ligno-cellulosic material through ester bonds. Preferably the cross-linking acid is a carboxylic acid having at least two carboxyl groups, even more preferably at least three carboxyl groups. The polyol preferably has at least six hydroxyl groups. The hydrophobic agent, which preferably is selected from a range of natural oils and waxes, is present within the ligno-cellulosic material showing a cross-linked structure. The hydrophobic agent is fixated within the treated section of the substrate by the cross-linking reaction that has taken place between the cross-linking acid, the polyol and the hydroxyl groups of the ligno-cellulosic material in the presence of the hydrophobic agent.

The resulting functionalised ligno-cellulosic material as described above will have significantly enhanced moisture and liquid resistance and further increased dimensional stability when compared to the untreated material. Additionally, the material obtained in the process of the invention shows improved anti-microbial properties and is fully recyclable together with similar untreated materials.

### Stabilisation, Hydrophobation and Enhanced Durability Treatment of a Porous or Low-Weight Ligno-Cellulosic Material

A first aspect of the present disclosure relates to a method for stabilisation, hydrophobation and enhanced durability treatment of a porous or low weight ligno-cellulosic material. The method for treating low-weight or porous materials is especially preferred in a method for the manufacturing of packaging materials or products intended for short term use during storage and transportation.

In a first embodiment of the first aspect of the present disclosure, the method is a method for stabilisation, hydrophobation and enhanced durability treatment of a porous or low-weight ligno-cellulosic material, wherein:
a cross-linking formulation comprising at least one cross-linking acid and at least one polyol is prepared in a base of water at a temperature below 120° C. and is applied to a porous or low-weight lignocellulosic material,
a hydophobation emulsion comprising at least one hydrophobic agent and a non-ionic surfactant is prepared separately from the cross-linking formulation and applied to the porous or low-weight ligno-cellulosic material, the ligno-cellulosic material being pre-shaped or in final product shape,
the chemically treated material is subjected to a curing step at a temperature initiating an esterification reaction between the cross-linking acid, the hydroxyl groups of the ligno-cellulosic material and the polyol, thus also fixating the hydrophobic agent within the cross-linked structure.

In a second embodiment of the first aspect of the present disclosure, the method is a method according to the first embodiment of the first aspect of the present disclosure, wherein said at least one cross-linking acid is selected from a range of carboxylic acids having at least two carboxyl groups, preferably the cross-linking acid is selected from 1-hydroxypropane-1,2,3-tricarboxylic acid, propane-1,2,3-tricarboxylic acid, 2-hydroxynonadecane-1,2,3-tricarboxylic acid, 2-hydroxypropane-1,2,3-tricarboxylic acid, benzene-1,3,5-tricarboxylic acid and prop-1-ene-1,2,3-tricarboxylic acid.

In a third embodiment of the first aspect of the present disclosure, the method is a method according to the first embodiment of the first aspect of the present disclosure, wherein said at least one polyol is selected from xylitol, sorbitol and erythritol.

In a fourth embodiment of the first aspect of the present disclosure, the method is a method according to any one of the embodiments 1-3 of the first aspect of the present disclosure, wherein said hydrophobation emulsion comprises at least one hydrophobic agent including at least one substance selected from fatty acid esters, fatty alcohols and pentacyclic triterpenoids, such as oleanolic acid, betulin and betulinic acid.

In a fifth embodiment of the first aspect of the present disclosure, the method is a method according to any one of the embodiments 1˜4 of the first aspect of the present disclosure, wherein said hydrophobation emulsion comprises at least one hydrophobic agent selected from a range of natural oils and waxes, preferably the hydrophobic agent is carnauba wax.

In a sixth embodiment of the first aspect of the present disclosure, the method is a method according to any one of the embodiments 1-5 of the first aspect of the present disclosure, wherein the cross-linking formulation and the hydrophobation emulsion are blended prior to application onto the ligno-cellulosic material.

In a seventh embodiment of the first aspect of the present disclosure, the method is a method according to any one of the embodiments 1-6 of the first aspect of the present disclosure, wherein the cross-linking formulation and the hydrophobation emulsion are added separately, optionally using different techniques.

In an eighth embodiment of the first aspect of the present disclosure, the method is a method according to any one of the embodiments 1-7 of the first aspect of the present disclosure, wherein the cross-linking formulation, the hydrophobation emulsion or the mix thereof is applied in liquid form, preferably by submersion, spraying or curtain coating.

In a ninth embodiment of the first aspect of the present disclosure, the methods is a method according to any one of the embodiments 1-8 of the first aspect of the present disclosure, wherein the cross-linking formulation, the hydrophobation emulsion or the mix thereof is applied to the ligno-cellulosic material at a temperature from 60° C. to 119° C., more preferably from 80° C. to 100° C., even more preferably from 90° C. to 95° C.

In a tenth embodiment of the first aspect of the present disclosure, the method is a method according to any one of the embodiments 1-9 of the first aspect of the present disclosure, the method being a method for the manufacturing of packaging materials or products intended for short term use during storage and transportation.

A second aspect of the present disclosure relates to a product of porous or low-weight ligno-cellulosic material. An especially preferred product of porous or low-weight ligno-cellulosic material is a recyclable single-use packaging product.

In a first embodiment of the second aspect of the present disclosure, the product is a product of porous or low-weight ligno-cellulosic material, wherein the ligno-cellulosic material comprises moieties of at least one polyol and at least one organic cross-linking acid being at least partially cross-linked to the hydroxyl groups of a ligno-cellulosic material through ester bonds, whereby the cross-linking acid is a carboxylic acid having at least two carboxyl groups, and wherein at least one hydrophobic agent selected from a range of natural oils and waxes is fixated within the ligno-cellulosic material by the cross-linked structure.

In a second embodiment of the second aspect of the present disclosure, the product is a product of porous or low-weight ligno-cellulosic material according to the first embodiment of the second aspect of the present disclosure, wherein said at least one hydrophobic agent includes at least one substance selected from fatty acid esters, fatty alcohols and pentacyclic triterpenoids, such as oleanolic acid, betulin and betulinic acid.

In a third embodiment of the second aspect of the present disclosure, the product is a product of porous or low-weight ligno-cellulosic material according to the first or second embodiment of the second aspect of the present disclosure, wherein said hydrophobic agent is carnauba wax.

In a fourth embodiment of the second aspect of the present disclosure, the product is a product of porous or low-weight ligno-cellulosic material according to any one of the embodiments 1-3 of the second aspect of the present disclosure, wherein the entire cross-section of the product shows a cross-linked structure.

In a fifth embodiment of the second aspect of the present disclosure, the product is a product of porous or low-weight ligno-cellulosic material according to any one of embodiments 1-4 of the second aspect of the present disclosure, wherein the product is a recyclable single-use packaging product.

In a sixth embodiment of the second aspect of the present disclosure, the product is a product of porous or low-weight ligno-cellulosic material according to the first embodiment of the second aspect of the present disclosure, wherein the bio-composite material is obtained by the method of the first embodiment of the first aspect of the present disclosure.

### Stabilisation, Hydrophobation and Enhanced Durability Treatment of a Solid Ligno-Cellulosic Material or Similar Density Ligno-Cellulosic Material

A third aspect of the present disclosure relates to a method for stabilisation, hydrophobation and enhanced durability treatment of a solid ligno-cellulosic material or similar density ligno-cellulosic material. The method for treating solid or similar density products is especially preferred for treatment of products or material intended for long-term use, such as construction materials.

In a first embodiment of the third aspect of the present disclosure, the method is a method for stabilisation, hydrophobation and enhanced durability treatment of a solid ligno-cellulosic material or similar density ligno-cellulosic material, wherein:
a cross-linking formulation comprising at least one carboxylic acid having at least two carboxyl groups as cross-linking acid and at least one polyol is prepared in a base of water at a temperature below 120° C.,
a hydophobation emulsion comprising at least one hydrophobic agent and a nonionic surfactant is prepared separately from the cross-linking formulation,
the cross-linking formulation and the hydrophobation emulsion are blended to form a mix,
the mix is applied to a pre-shaped or final product shape of solid or similar density ligno-cellulosic material, and
the chemically treated material is subjected to a curing step at a temperature initiating an esterification reaction between the cross-linking acid, the polyol and the hydroxyl groups of the ligno-cellulosic material and thus also fixating the hydrophobic agent.

In a second embodiment of the third aspect of the present disclosure, the method is a method according to the first embodiment of the third aspect of the present disclosure, wherein said at least one cross-linking acid is a tricarboxylic acid, preferably the cross-linking acid is selected from 1-hydroxypropane-1,2,3-tricarboxylic acid, propane-1,2,3-tricarboxylic acid, 2-hydroxynonadecane-1,2,3-tricarboxylic acid, 2-hydroxypropane-1,2,3-tricarboxylic acid, benzene-1,3,5-tricarboxylic acid and prop-1-ene-1,2,3-tricarboxylic acid.

In a third embodiment of the third aspect of the present disclosure, the method is a method according to the first embodiment of the third aspect of the present disclosure, wherein said at least one polyol is selected from xylitol, sorbitol and erythritol.

In a fourth embodiment of the third aspect of the present disclosure, the method is a method according to any one of the embodiments 1-3 of the third aspect of the present disclosure, wherein said hydrophobation emulsion comprises at least one hydrophobic agent including at least one substance selected from fatty acid esters, fatty alcohols and pentacyclic triterpenoids, such as oleanolic acid, betulin and betulinic acid.

In a fifth embodiment of the third aspect of the present disclosure, the method is a method according to any one of embodiments 1-4 of the third aspect of the present disclosure, wherein said hydrophobation emulsion comprises at least one hydrophobic agent selected from a range of natural oils and waxes, preferably the hydrophobic agent is carnauba wax.

In a sixth embodiment of the third aspect of the present disclosure, the method is a method according to any one of the embodiments 1-5 of the third aspect of the present disclosure, wherein the mix of cross-linking formulation and the hydrophobation emulsion is applied in liquid form, preferably by submersion, spraying or curtain coating.

In a seventh embodiment of the third aspect of the present disclosure, the method is a method according to any one of the embodiments 1-6 of the third aspect of the present disclosure, wherein the mix of cross-linking formulation and the hydrophobation emulsion is applied in a pressurised environment through impregnation in liquid for or as a mist in semi-gaseous or atomised form.

In an eighth embodiment of the third aspect of the present disclosure, the method is a method according to any one of the embodiments 1-7 of the third aspect of the present disclosure, wherein the mix of cross-linking formulation and the hydrophobation emulsion is applied to the ligno-cellulosic material at a temperature from 60° C. to 119° C., more preferably from 80° C. to 100° C., even more preferably from 90° C. to 95° C.

In a ninth embodiment of the third aspect of the present disclosure, the method is a method according to any one of embodiments 1-8 of the third aspect of the present disclosure, wherein the uptake of the mix of cross-linking formulation and the hydrophobation emulsion is enhanced by use of microwave treatment.

A fourth aspect of the present disclosure relates to a solid ligno-cellulosic material or similar density ligno-cellulosic material combined into a composite. An especially preferred product of solid or similar density ligno-cellulosic material is a construction material, a construction product, an interior or exterior building product, a furniture product, a transportation product or a storage product.

In a first embodiment of the fourth aspect of the present disclosure, the product is a product of solid ligno-cellulosic material or similar density ligno-cellulosic material combined into a composite, wherein the product has at least a surface of ligno-cellulosic material comprising moieties of at least one polyol and at least one organic cross-linking acid being at least partially cross-linked to the hydroxyl groups of a ligno-cellulosic material through ester bonds, whereby the cross linking acid is a carboxylic acid having at least two carboxyl groups, and wherein a hydrophobic agent selected from a range of natural oils and waxes is present within the ligno-cellulosic material showing a cross-linked structure.

In a second embodiment of the fourth aspect of the present disclosure, the product is a product according to the first embodiment of the fourth aspect of the present disclosure, wherein said at least one hydrophobic agent includes at least one substance selected from fatty acid esters, fatty alcohols and pentacyclic triterpenoids, such as oleanolic acid, betulin and betulinic acid.

In a third embodiment of the fourth aspect of the present disclosure, the product is a product according to the first or second embodiment of the fourth aspect of the present disclosure, wherein said hydrophobic agent is carnauba wax.

In a fourth embodiment of the fourth aspect of the present disclosure, the product is a product according to any one of the embodiments 1-3 of the fourth aspect of the present disclosure, wherein the product is a construction material, a construction product, an interior or exterior building product, a furniture product, a transportation product, or a storage product.

In a fifth embodiment of the fourth aspect of the present disclosure, the product is a product according to any one of the embodiments 1-3 of the fourth aspect of the present disclosure, the product being selected from a group of products comprising structural wood elements, cross laminated timber, glue laminated beams, laminated veneer lumber, plywood, medium and high density fibreboard, particle and strand boards, decking boards, flooring, cladding, wall boards, general construction products produced from ligno-cellulosic materials, transport crates and pallets.

In a sixth embodiment of the fourth aspect of the present disclosure, the product is a product according to the first embodiment of the fourth aspect of the present disclosure, wherein the bio-based ligno-cellulosic material surface is obtained by the method according to the first embodiment of the third aspect of the present disclosure.

### EXAMPLES

### Example 1: Treatment of Cardboard

A hydrophobation emulsion was prepared by emulsifying carnauba wax in an amount of 6 wt-% in water at a temperature ranging from about 95° C. to 100° C. Cremophor® RH 40 from BASF was used as surfactant. A cross-linking formulation was prepared at a similar temperature by dissolving citric acid and sorbitol in a 3:1 ratio in water to a total solids content of 20%. The functional emulsion and the cross-linking formulation were mixed at a similar temperature to form a uniform formulation and a pulp sheet material of 20 mm×100 mm was soaked in the mixed formulation at a temperature of from about 95° C. to 100° C. for 1 min. The pulp sheet material was dried at 100° C. for 1 hour leading to an average WPG of 75%. The final curing was performed at 180° C. for 15 min.

The hydrophobation treatment significantly increased the wetting contact angle of the material and the moisture resistance. After a 5 min water soaking test at 23° C. the WPG of the cardboard piece was 4.4%, calculated as an average value for five test pieces.

As reference, an untreated and uncured cardboard piece having the same dimensions showed a WPG of 190% in the same water soaking test. Curing of the untreated cardboard resulted in a slightly better water resistance, as the WPG after the 5 min soaking test was 122.6%. Even the cured cardboard thus showed a water uptake that was around 28 times higher than the water uptake for the cardboard treated according to the present invention.

### Example 2: Treatment of Solid Wood

A first solution of citric acid, sorbitol and water with solids content of 15% solids to water ratio was prepared via heated mixing synthesis at a temperature above 90° C. A second solution consisting of carnauba wax in a ratio of 10% by weight of the combined first solution and an industrially available nonionic surfactant, which was added in a ratio of 10% of the wax component by weight, was prepared separately by mixing synthesis at a temperature above 90° C. Upon completion of the two solutions the second solution was blended to the first solution at a temperature above 90° C. and further synthesized until fully blended into a homogenous emulsion.

After blending, solid wood pine samples measuring 10×10×50 mm were soaked in the emulsion at a temperature around 95° C. for a period of 15 min. Samples were weighed to determine the weight percentage gain compared to reference sample. Treated samples were then dried in a conventional oven for 45 min at a constant temperature of 103° C., after which they were dried to determine the solids content weight gain. After this step some of the samples were taken to an oven at a temperature of 180° C. for 30 min to carry out the curing reaction and further weighed after for final weight.

Water soaking trials were conducted on the obtained test pieces treated with the solution containing citric acid, sorbitol and carnauba wax. The samples were named CASC1, CASC2 and CASC3, of which CASC1 and CASC2 were cured and CASC3 uncured. All samples, including a reference sample (REF Pine) of untreated solid wood, were applied to a water soaking bath (room temperature conditions 23° C.) where they freely soaked with no end grain sealants and then measured for weight gain after 1 hours, 3 hours and 5 hours. The results of the soaking test are presented in Table 1.

| Sample | WPG % 1 hr | WPG % 3 hr | WPG % 5 hr |
|---|---|---|---|
| CASC1 Cured | 7.27 | 10.61 | 14.55 |
| CASC2 Cured | 7.86 | 10.38 | 16.35 |
| CASC3 no curing | 16.80 | 30.40 | 43.73 |
| REF Pine | 57.99 | 57.99 | 62.45 |

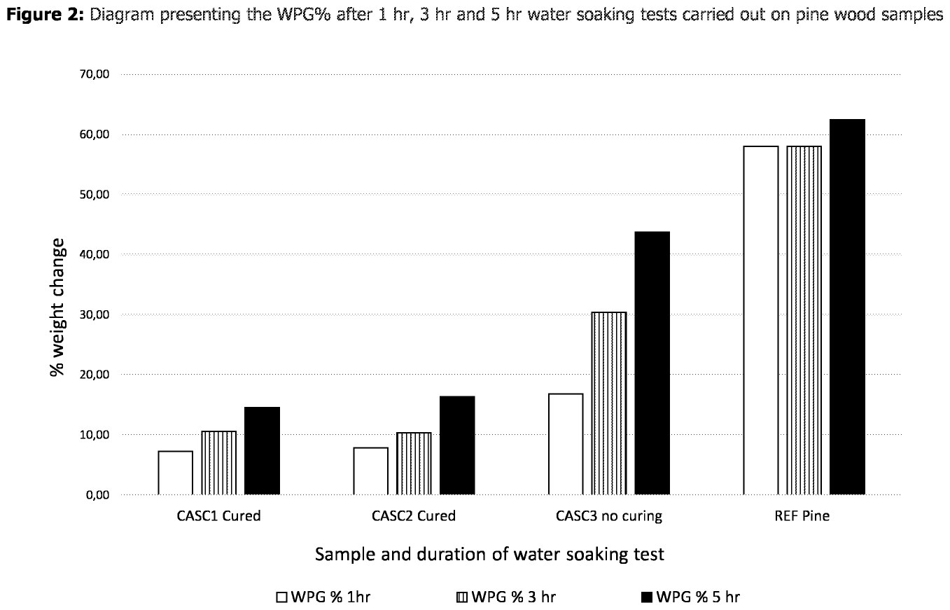

As can be seen from the results presented in Table 1 and the diagram of FIG. 2, the trial succeeded in significantly reducing the water uptake into the treated pine samples, with even greater reduction in moisture uptake in the samples which also had the curing step applied and even more significantly compared to the untreated reference material. The results proves that such a formulation of the emulsion and treatment could provide a wood product which will become far less suspectable to shorter term water exposure, such as during the construction of large wood buildings during period of rain, whereby water damage may normally occur, or for wooden packaging crates and boxes which may be exposed to periodic rain exposure.

1-31. (canceled)

32. Method for stabilisation, hydrophobation and enhanced durability treatment of a solid ligno-cellulosic material or similar density ligno-cellulosic material, wherein:
a cross-linking formulation comprising at least one carboxylic acid having at least two carboxyl groups as cross-linking acid and at least one polyol is prepared in a base of water at a temperature below 120° C.,
a hydrophobation emulsion comprising at least one hydrophobic agent and a non-ionic surfactant is prepared separately from the cross-linking formulation,
the cross-linking formulation and the hydrophobation emulsion are blended to form a mix,
the mix is applied to a pre-shaped or final product shape of solid or similar density ligno-cellulosic material, and
the chemically treated material is subjected to a curing step at a temperature initiating an esterification reaction between the cross-linking acid, the polyol and the hydroxyl groups of the ligno-cellulosic material and thus also fixating the hydrophobic agent.

33. The method according to claim 32, wherein said at least one cross-linking acid is a tricarboxylic acid.

34. The method according to claim 32, wherein said at least one polyol is selected from xylitol, sorbitol and erythritol.

35. The method according to claim 32, wherein said hydrophobation emulsion comprises at least one hydrophobic agent including at least one substance selected from fatty acid esters, fatty alcohols and pentacyclic triterpenoids.

36. The method according to claim 32, wherein said hydrophobation emulsion comprises at least one hydrophobic agent selected from a range of natural oils and waxes.

37. The method according to claim 32, wherein the mix of cross-linking formulation and the hydrophobation emulsion is applied in liquid form.

38. The method according to claim 32, wherein the mix of cross-linking formulation and the hydrophobation emulsion is applied in a pressurised environment through impregnation in liquid for or as a mist in semi-gaseous or atomised form.

39. The method according to claim 32, wherein the mix of cross-linking formulation and the hydrophobation emulsion is applied to the ligno-cellulosic material at a temperature from 60° C. to 119° C.

40. The method according to claim 32, wherein the uptake of the mix of cross-linking formulation and the hydrophobation emulsion is enhanced by use of microwave treatment.

41. Product of solid ligno-cellulosic material or similar density ligno-cellulosic material combined into a composite, wherein the product has at least a surface of ligno-cellulosic material comprising moieties of at least one polyol and at least one organic cross-linking acid being at least partially cross-linked to the hydroxyl groups of a ligno-cellulosic material through ester bonds, whereby the cross linking acid is a carboxylic acid having at least two carboxyl groups, and wherein a hydrophobic agent selected from a range of natural oils and waxes is present within the ligno-cellulosic material showing a cross-linked structure.

42. The product according to claim 41, wherein said at least one hydrophobic agent includes at least one substance selected from fatty acid esters, fatty alcohols and pentacyclic triterpenoids.

43. The product according to claim 41, wherein said hydrophobic agent is carnauba wax.

44. The product according to claim 41, wherein the product is a construction material, a construction product, an interior or exterior building product, a furniture product, a transportation product, or a storage product.

45. The product according to claim 41, the product being selected from a group of products comprising structural wood elements, cross laminated timber, glue laminated beams, laminated veneer lumber, plywood, medium and high density fibreboard, particle and strand boards, decking boards, flooring, cladding, wall boards, general construction products produced from ligno-cellulosic materials, transport crates and pallets.

46. Product of solid ligno-cellulosic material or similar density ligno-cellulosic material combined into a composite, wherein the product has at least a surface of ligno-cellulosic material comprising moieties of at least one polyol and at least one organic cross-linking acid being at least partially cross-linked to the hydroxyl groups of a ligno-cellulosic material through ester bonds, whereby the cross linking acid is a carboxylic acid having at least two carboxyl groups, and wherein a hydrophobic agent selected from a range of natural oils and waxes is present within the ligno-cellulosic material showing a cross-linked structure, wherein the bio-based ligno-cellulosic material surface is obtained by the method of claim 32.

47. The method of claim 33, wherein said at least one cross-linking acid is selected from the group consisting of 1-hydroxypropane-1,2,3-tricarboxylic acid, propane-1,2,3-tricarboxylic acid, 2-hydroxynonadecane-1,2,3-tricarboxylic acid, 2-hydroxypropane-1,2,3-tricarboxylic acid, benzene-1,3,5-tricarboxylic acid and prop-1-ene-1,2,3-tricarboxylic acid.

48. The method of claim 36, wherein said hydrophobation emulsion comprises carnauba wax.

49. The method of claim 37, wherein the mix of cross-linking formulation and the hydrophobation emulsion is applied by submersion, spraying or curtain coating.

50. The method of claim 39, wherein the mix of cross-linking formulation and the hydrophobation emulsion is applied to the ligno-cellulosic material at a temperature from 80° C. to 100° C.

51. The method of claim 39, wherein the mix of cross-linking formulation and the hydrophobation emulsion is applied to the ligno-cellulosic material at a temperature from 90° C. to 95° C.
